(19) United States
(12) Patent Application Publication
(10) Pub. No.: US 2012/0325152 A1
(43) Pub. Date: Dec. 27, 2012

(54) METHODS AND COMPOSITIONS FOR NEMATODE GROWTH

(76) Inventors: Christopher E. Hopkins, Salt Lake City, UT (US); John P. Manfredi, Salt Lake City, UT (US)

(21) Appl. No.: 13/531,039

(22) Filed: Jun. 22, 2012

Related U.S. Application Data
(60) Provisional application No. 61/500,091, filed on Jun. 22, 2011.

(51) Int. Cl. A01K 67/033; A23L 1/48

(52) U.S. Cl. 119/6.7; 800/8; 800/13; 426/132

(57) ABSTRACT

Compositions for nematode growth and methods for using the same for nematode viability are described. The compositions contain a solid matrix material that is dispersed in a liquid medium. The solid matrix can be immersed, bathed, and/or interspersed in a liquid medium to form a composite culture that is capable of maintaining or growing nematodes at unexpectedly high densities and/or with nominal stress pathway activity. Other embodiments are described.

### CROSS-REFERENCE TO RELATED APPLICATION

This application claims priority of U.S. provisional patent application 61/500,091 filed Jun. 22, 2011, the entire disclosure of which is incorporated herein by reference.

### FIELD

This application describes methods and compositions for nematode growth and viability. More specifically, this application describes compositions containing solid matrices in combination with liquid media and their use for nematode growth and viability.

### BACKGROUND

Nematodes like Caenorhabditis elegans are cultured, grown, or maintained in the laboratory setting in various formats including liquid media cultures and on agarose plates. Both the liquid and agarose formats lack specific qualities that make them less than optimal for certain applications. On solid surfaces such as agarose, nematodes use an undulatory motion that allows them to move efficiently towards nutrients e.g., food. In liquids, on the other hand, nematodes exhibit a thrashing movement that provides negligible locomotion yet consumes much energy. Given their inability to move productively in liquids, nematodes sink to the bottom of a column of static liquid, where, depending on the fluid depth, the density of the worms, and their oxygen consumption, hypoxic or virtual anoxic conditions can exist. The resulting adverse effects on the nematodes can include physiological stress, retarded development, and morbidity, all of which compromise or severely limit the use of nematodes in experimental formats that require liquid media.

The pharmaceutical, biotechnology, and academic research communities have benefited enormously from the development of liquid-handling equipment and instrumentation. These liquid-handling technologies, which often contain automated and robotic features, have enabled scientists to routinely conduct experimentation on a scale that would otherwise be impractical. While attempts have been made to apply liquid-handling capabilities to nematode research, efforts have been handicapped by difficulties growing healthy and unstressed nematodes in liquid culture. This handicap can be severe in the case of nematodes that are unable to move normally.

### SUMMARY

This application relates to compositions for nematode growth and methods for using the same for nematode viability. The compositions (or composite culture composition) contain a solid matrix (or solid matrices) that is dispersed in a liquid medium (or liquid media). The solid matrices can be used to culture (grow, promote the growth of, maintain, or promote the survival of) nematodes in liquid (e.g., aqueous) culture-like conditions. Two distinct and combinable liquid media formats that allow for surprisingly robust culturing, growth, or maintenance of nematodes can be used. In some embodiments, a solid matrix material can be immersed, bathed, and/or interspersed in a liquid medium to form a composite culture that is capable of maintaining or growing nematodes at unexpectedly high densities and/or with nominal stress pathway activity. The solid matrix material can comprise a plurality of particles that when settled in a container or vessel has voids containing liquid media between the individual particles that allow for the nematodes to move through these void spaces and in between the particles of the solid matrix. These particles can be non-porous or can be porous such that the pores are of sufficient size to allow the nematodes to move through the particle or around the particle having pores. Alternatively, the particles may be porous, but the size of the pores do not allow for the nematodes to move through the particles. A culture system can be provided which comprises a gas-permeable floor (e.g., mesh or membrane) upon which sits a liquid medium such that the liquid medium does not pass through the gas-permeable floor (or the gas-permeable floor is substantially leak-free e.g., lets little or negligible amounts of the liquid medium to pass through the floor over the course of an experimental or desired time frame).

Solid Matrix Material

In some embodiments, the solid matrix material comprises a plurality of particles having a density greater than the liquid media. Since the density of the liquid medium can range between about 0.8 g/cm³ to about 1.2 g/cm³ (or can be about 1.0 g/cm³), the particles have a density greater than those amounts. Each particle can have a shape such that when the plurality of particles is immersed, bathed or interspersed in the liquid medium, allows for the movement of nematodes in the liquid medium and around the solid matrix material. The solid matrix material particles can be porous or non-porous and the particles can be spherical or can be beads which, in some configurations, can be derivatized. The solid matrix material can also be granular. Where the solid matrix material comprises a plurality of spheres, each sphere can have a diameter from about 0.01 mm to about 10 mm. The solid matrix material can have anywhere from about 2 to 10 million particles. The solid matrix can be comprised of granular particles having a US mesh size in the range of about 3.5 to about 632. The solid matrix material can be made of a material selected from plastic, sand, diatomaceous earth, silicon, glass, inert carbon, silica, zirconia, or combinations thereof.

Liquid Medium

In some embodiments, any liquid medium (or liquid media) can be used that allows for surprisingly robust culturing, growth, or maintenance of nematodes. The liquid medium can comprise a buffer, such as any phosphate buffer. The liquid media can have components sufficient for growing or maintaining a plurality of nematodes. The liquid media can have a nutrient which is an alive or dead bacterial preparation. In one aspect, the liquid medium can have a nutrient which is inactivated bacteria. The amount of liquid media used is such that when mixed together and settled in a receptacle, the liquid media completely covers the solid matrix material and is no greater than about 2 times the height of the solid matrix material. In one aspect, the liquid media completely covers the solid matrix material such that about 10% or less of the liquid is above the solid matrix material.

Container Composition or Object

In some embodiments, the composition with the solid matrix (or solid matrices) and the liquid medium (or liquid media) is contained in an object or a container. The container (or object) can have one or more receptacles where at least one (or more) of the receptacles contains a plurality of nematodes, the solid matrix material, the liquid media, or any combination thereof. In some aspects, at least 1 receptacle of the container can comprise a plurality of nematodes. In some aspects, the container comprises anywhere from 2 to 10,000 receptacles. In some aspects, the container can be a microtiter plate having from 6 wells to 1536 wells. In some aspects, the object or container has a solid matrix material in at least about 10% of the receptacles. In some aspects, the container can be an Eppendorf tube, a 0.6 ml microtube, a 1.5 ml microtube, a PCR tube, PCR tube strips, a glass test tube, a plastic test tube, or combinations thereof.

Bioreactor

In some embodiments, a bioreactor is used to grow and/or culture nematodes. The bioreactor can contain at least one solid matrix material immersed, interspersed, or bathed in a liquid medium. The liquid medium contains at least one buffering agent, including a phosphate buffer. The bioreactor can contain a plurality of nematodes. The bioreactor can have a temperature regulation device to maintain and control the temperature of the bioreactor. The bioreactor can have at least one inlet port, at least one outlet port, and a flow regulator that allows for and controls the passage of a liquid (e.g., aqueous) through the bioreactor. The bioreactor can be configured to collect material produced (e.g., excreted or secreted) by the plurality of nematodes (e.g., protein, antibody, or other product) and/or the bioreactor can be configured to collect the plurality of nematodes. In some configurations, the bioreactor has the capacity to contain a volume of about 10 mL or more of composite culture composition. In one aspect, the bioreactor has the capacity to contain—or actually contains—a volume of about 1 L or more, about 5 L or more, or about 10 L or more of composite culture composition.

Method of Use for Culturing Nematodes

In some embodiments, methods can be used for growing or maintaining a plurality of nematodes. These methods contact a plurality of nematodes with the composite culture composition. The composite culture composition can comprise a solid matrix material and an aqueous based buffer having one or more nutrients and is kept under conditions sufficient to promote the growth of or maintain a plurality of nematodes. These conditions can include maintaining the composite culture composition at a substantially constant temperature of anywhere from about 10° C. to about 29° C. The plurality of nematodes can include anywhere from 2 to 100,000 nematodes. The ages of the plurality of nematodes can be synchronized or unsynchronized. The nematodes can be wild-type C. elegans or genetically engineered C. elegans.

In other embodiments, methods can be used for improving nematode viability in liquid culture. These methods contact a nematode or population of nematodes in need of improved viability in liquid culture with the composite culture composition (containing the liquid culture medium having a solid matrix material). The nematode can be a genetically engineered nematode. The nematode can have a reduced viability wherein the reduced viability is induced by or results from a mutation, an external agent, or both. The external agent can be a drug, a toxin, a pharmaceutical preparation, an environmental poison, or combinations thereof.

Assay Methods

In some embodiments, the solid matrix material can be used in assay methods. These methods comprise providing a solid matrix material immersed, bathed, interspersed in or combined with a liquid medium and treating a nematode or plurality of nematodes with an agent and introducing the nematode or plurality of nematodes treated with the agent to the solid matrix material immersed in or combined with the liquid medium. These methods can also comprise introducing a nematode or plurality of nematodes to the solid matrix material immersed in or combined with liquid medium and treating the nematode culture with an agent. These methods can also comprise maintaining or growing the nematode culture for a time sufficient to allow for the agent to affect the nematode culture or to allow the growth of the nematode culture. The time sufficient can be at least 1 hour or at least one day. These methods can also comprise determining, monitoring or measuring the effect of the agent on the nematode culture after it has been maintained for a time sufficient to allow for the agent to affect the nematode culture or allow the nematode culture to grow.

Kit

In some embodiments, a kit can be used for nematode growth, maintenance or culturing. In these embodiments, the kit comprises an object or container having a plurality of receptacles. The kit can have any quantity of solid matrix material. The kit can also have a liquid media comprising at least one buffering agent, such as a phosphate-based buffer. The liquid media can also be sterile. The kit can comprise one or more nutrients. The nutrient or the liquid media can be configured to be reconstituted by the addition of water or sterile water. The kit can be configured to contain water or sterile water. The kit can contain a device for dispensing or measuring a unit volume of solid matrix material, nutrient, liquid media, or water wherein the unit volume is the amount sufficient for each receptacle of the object or container. The kit can comprise a plurality of container or receptacles wherein each receptacle or container comprises an amount of the solid matrix material. The solid matrix material in the kit can be glass beads having a diameter of between 0.05 mm to 0.5 mm. The kit can contain a plurality of containers or receptacles having gas-permeable floors.

Nematode Viability

In some embodiments, methods can be used to culture (grow, obtain, maintain, promote the growth of, or promote the survival of) or improve the viability of nematodes in liquid media when the nematodes are locomotion-compromised. These methods contact a locomotion-compromised nematode or population of nematodes with a liquid medium in a vessel, container or receptacle having a gas-permeable floor, e.g., wherein the floor is a gas-permeable mesh (or sieve) or membrane. The nematodes can be wild-type with depressed mobility or locomotion (e.g., by treatment of the nematodes or cultures thereof with an agent). The nematodes can be slowly moving or sedentary nematodes. The nematodes can carry a genetic mutation (or mutations) that can interfere with or depresses mobility. In one aspect, the liquid medium can have sufficient viscosity to depress mobility or locomotion of the nematodes. The floor can comprise a mesh that allows gas but not liquid to cross the floor substantially or spontaneously. The mesh floor can be in the range of 10 to 200 micron mesh, including a gas-permeable floor with a 40 micron mesh (US mesh size 325) that can be crossed by nematodes at the L1 stage of development but not at the L4 or adult stage of development. The gas-permeable floor can also comprise a mesh through which nematodes at a particular stage of development can pass when the vessel is centrifuged. The gas-permeable floor can contain a membrane that allows gas but not liquid to flow substantially or spontaneously. In one aspect, the gas-permeable floor comprises a membrane that allows passage of select gases but not others, for example a membrane that allows passage of carbon dioxide and oxygen but no other gases. The vessel can be a microtiter plate where the floors of the wells are gas-permeable. The vessel can also comprise a well of a 96-well microtiter plate that has a floor comprising a 40 micron nylon mesh. The microtiter plate can comprise a gas permeable floor that is configured to fit into or sit upon a receiver plate.

### BRIEF DESCRIPTION OF THE DRAWINGS

The following description can be better understood in light of the Figures, in which:

FIGS. 1A and 1B shows a schematic representation of some embodiments of a container with a composite culture composition;

FIGS. 2A and 2B shows a schematic representation of some embodiments of a microtiter plate format;

FIG. 3 shows a schematic representation of some embodiments of an exemplary bioreactor;

FIG. 4 shows a schematic representation of some embodiments of a gas-permeable floor;

FIG. 5 shows some embodiments of the effects of a solid matrix of glass beads and E. coli on worm stress in liquid media;

FIG. 6 shows some embodiments of the effect of beads on E. coli tolerance in liquid growth conditions; and

FIG. 7 shows some embodiments on the growth comparisons of paralyzed nematodes in liquid and on solid media.

The Figures illustrate specific aspects of the composite culture composition and methods for using such compositions. Together with the following description, the Figures demonstrate and explain the principles of the methods and compositions produced through these methods. In the drawings, the thickness of layers and regions are exaggerated for clarity. The same reference numerals in different drawings represent the same element, and thus their descriptions will not be repeated. As the terms “on”, “attached to”, or “coupled to” are used herein, one object (e.g., a material, a layer, a substrate, etc.) can be on, attached to, or coupled to another object regardless of whether the one object is directly on, attached, or coupled to the other object or there are one or more intervening objects between the one object and the other object. Also, directions (e.g., above, below, top, bottom, side, up, down, under, over, upper, lower, horizontal, vertical, “x,” “y,” “z,” etc.), if provided, are relative and provided solely by way of example and for ease of illustration and discussion and not by way of limitation. In addition, where reference is made to a list of elements (e.g., elements a, b, c), such reference is intended to include any one of the listed elements by itself, any combination of less than all of the listed elements, and/or a combination of all of the listed elements.

### DETAILED DESCRIPTION

The following description supplies specific details in order to provide a thorough understanding. Nevertheless, the skilled artisan would understand that the composite culture composition and associated methods of making and using such compositions can be implemented and used without employing these specific details. Indeed, the composite culture composition and associated methods can be placed into practice by modifying the illustrated devices and methods and can be used in conjunction with any other apparatus and techniques conventionally used in the industry. For example, while the description refers to nematode growth, it could be modified to be used with other organisms outside of the phylum Nematoda that can experience stress when cultured in liquid media not having a solid matrix component or gas-permeable floor as described herein.

Some embodiments of the compositions and their use for nematode growth are described herein and illustrated in the Figures. These compositions and their associated methods provide for incredibly robust growth of nematodes, including wild-type nematodes and impaired nematodes. Culturing nematodes in some embodiments refers to the ability to grow, to obtain, to maintain, to promote growth, or to promote survival of nematodes. For example, for a given surface area of a plate or microtiter well up to 10-fold or more nematodes can be cultured or obtained using these methods and compositions. Alternatively, the stress induced by stimuli or an external agent can be significantly reduced in liquid media by addition of a solid matrix to that liquid media as described. As well, locomotion-depressed nematodes can be grown in liquid media in a similar fashion as on solid media (e.g., agarose) based on the use of a liquid medium that sits upon a gas-permeable floor. Thus, the compositions and methods can be used for growing, maintaining, or culturing nematodes and for improving nematode viability.

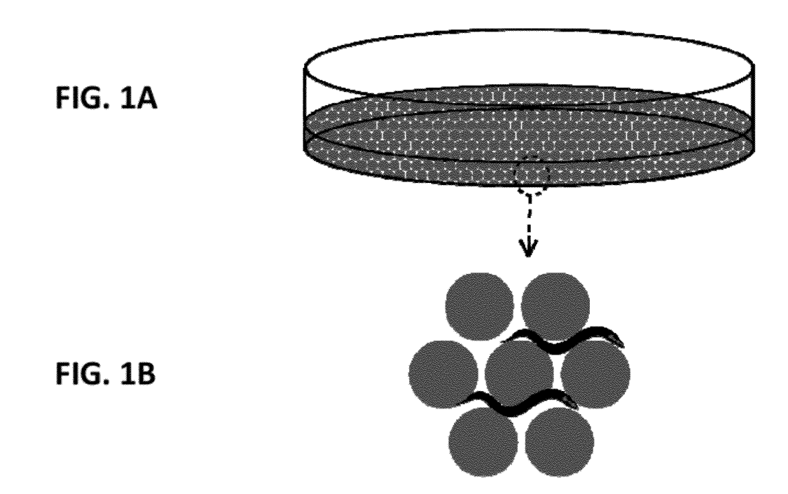

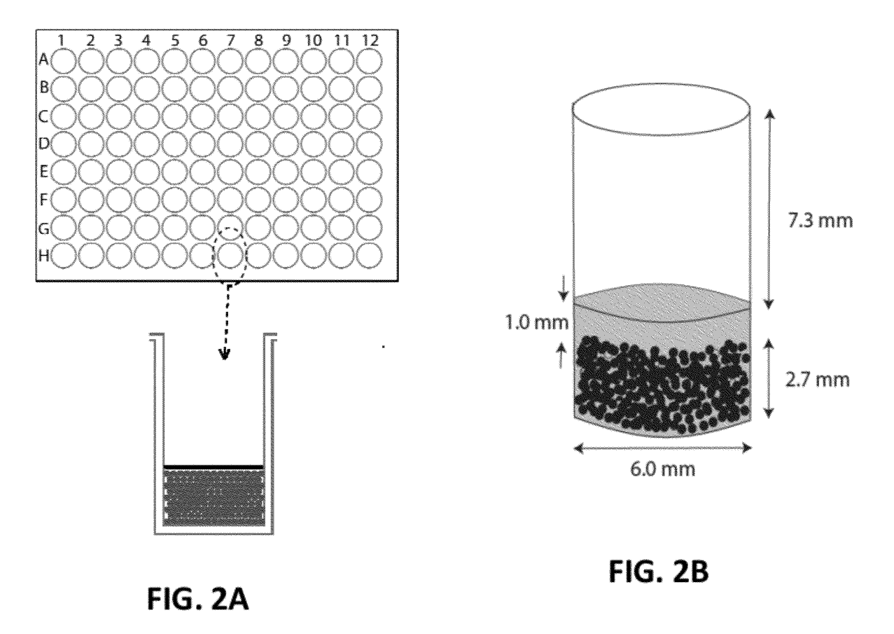

Several exemplary embodiments, not intended to limit the scope of the application, are illustrated in FIGS. 1 and 2 which show solid matrices, composite culture compositions, and containers having the solid matrices and composite culture compositions as described herein. When the liquid media is higher than the top of the column or level of the solid matrix material when settled in the container or object, the nematodes can experience undesirable stress. Analogously, if the level of the liquid medium is lower or much lower than the top of the column or level of liquid medium, the nematodes can also experience undesirable stress.

FIG. 1A illustrates a petri plate or dish having a composite culture medium (e.g., a plurality of glass beads and liquid medium). FIG. 1B shows a close-up view showing nematodes within the composite culture composition. FIG. 2A shows a 96-well microtiter plate with a close-up view of a well having a solid matrix material (e.g., spheres or beads) and liquid medium where the liquid medium is at a level in the well that is slightly above the top of the layer of solid matrix material (e.g., about 1 mm above the solid matrix material.) FIG. 2B is another close-up view of an individual well of a microtiter plate giving exemplary dimensions. The composite culture composition can be in a cylindrical well of a microtiter plate which has a diameter of 6 mm and a height of 11 mm. The solid matrix material (e.g., spheres or beads) fills the well to about a height of 2.7 mm, the composite culture composition (solid matrix plus liquid medium) fills the well to a height of about 3.7 mm, therefore giving a layer of about 1 mm of liquid medium above the top of the solid matrix material (bathed in liquid medium).

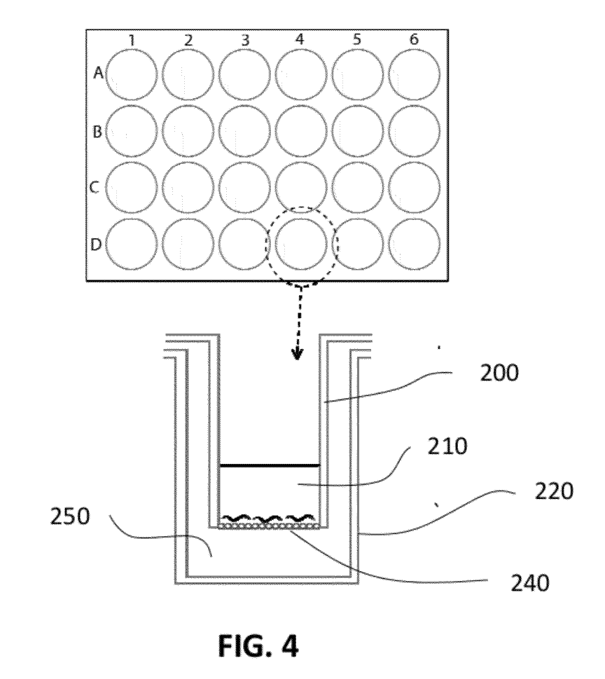

These compositions can be used for improving nematode viability in liquid or liquid cultures. Improving the nematode viability in some embodiments refers to the ability to reduce physiological stress, decrease retarded growth, increase nematode density, increase biomass, or improve morbidity. Up to about a 10-fold or more increase in nematode viability can be obtained using these compositions and methods. The combination of a solid matrix with a liquid medium or liquid culture improves nematode viability as estimated by nematode density, stress-induced death or stress-induced gene expression reporter assays (e.g., transgenic C. elegans having a heat shock protein promoter driving expression of a reporter gene (fluorescent protein)). Furthermore, the use of liquid media on top of a gas permeable floor can be advantageous for the liquid culture of nematodes, especially movement-compromised nematodes. The locomotion-depressed nematodes can grow in liquid culture with a gas-permeable floor similarly to how they grew with solid media. An exemplary embodiment of a gas-permeable floor system, not intended to limit the scope of this application, is illustrated in FIG. 4. FIG. 4 shows a well, 200, of a microtiter plate having a gas-permeable floor, 240 (e.g., membrane or mesh), a volume of liquid medium, 210, is contained within the well. The well, 200, is of a smaller length and diameter as compared to the receiver well, 220, which can have liquid or no liquid in the space it encloses, 250. Thus, the application relates to nematode culture systems for improving the availability of essential nutrients to the worms. A variety of configurations of the solid matrices and gas-permeable-floor culture systems are described in more detail below.

Adding or combining a solid matrix with a liquid medium increases or improves nematode health by various measurements including time to 1 mm (the time of growth required for the nematode or population of nematodes to reach 1 mm in length). Thus, in some embodiments, a solid matrix can be used that enables worms to move within a liquid medium to points at which growth conditions such as oxygen levels (or nutrients) are optimal or toxins are minimal. In one configuration, glass beads (about 0.1 mm in diameter) can be combined with bacteria-containing (e.g., inactivated bacteria-containing) aqueous media such that the top of the column of glass beads is 1 mm below the liquid surface. It is believed that the nematodes use the solid support of the glass bead matrix to move within the liquid medium to a local environment where they thrive. The matrix material contains spaces between the individual particles of the matrix that contains liquid media and allows for the nematodes to move through the matrix. In some embodiments, the matrix material can have pores within individual particles that allow the nematodes to move through. As conditions within the medium change, for example, as nutritional bacteria are consumed, oxygen is depleted, and metabolic waste is produced by the worms, the worms move to a more optimal local environment within the liquid medium. The glass beads enable worms to translocate not only in a horizontal plane through the liquid medium, but also along the oxygen gradient (or e.g., nutritional gradient) that varies with depth of the liquid medium (or other spatially defined region), enabling them to find the best available conditions for growth and survival. In other configurations, inert carbon can be used as the matrix to support movement of the animals within the liquid medium. Yet other configurations can use diatomaceous earth as the matrix. Thus, combining a solid matrix with a liquid medium can reduce or eliminate the adverse effects on nematodes of immersion in the liquid medium.

In some embodiments, a composition or object comprising a solid matrix material can be used for nematode growth. The solid matrix material can comprise a plurality of particles. Each particle can have a shape so that when the plurality of particles is immersed in or combined with liquid media, they allow for the movement of nematodes in the liquid media and around the solid matrix. The solid matrix material can be solid or porous. The particles of the solid matrix material can gave a shape selected from granular, spherical, cylindrical, polyhedron, or combinations there. In some aspects, the solid matrix can be spherical. In some aspects, the solid matrix material can be granular. In some aspects, the solid matrix material can be a bead or a derivatized bead.

The solid matrix material can comprise a material having a plurality of particles having a density that is greater than the liquid media. In some aspects, the density of the liquid media can be between 0.8 gram/cm³ to 1.2 gram/cm³. In other aspects, the density of the liquid media can be about 1.0 gram/cm³ since the liquid media can be water-based (e.g., aqueous) and therefore have a density similar to that of water or 1 g/cm³. Thus, the solid matrix material can have a density ranging from about 1.1 g/cm³ to about 5 g/cm³, about 1.2 g/cm³ to about 5 g/cm³, about 1.5 g/cm³ to about 4.5 g/cm³, about 1.7 g/cm³ to about 4.0 g/cm³, about 1.9 g/cm³ to about 3.5 g/cm³, about 2.1 g/cm³ to about 3.0 g/cm³, or about 2.3 g/cm³ to about 2.8 g/cm³. In other embodiments, the density of the solid matrix material can be any sub-range or combination of these amounts.

The solid matrix material can have any number of particles. In some embodiments, the solid matrix material can have a plurality of particles having from 2 to 10 million particles, 2 to 1 million particles, 10 to 500,000 particles, 25 to 250,000 particles, 50 to 200,000 particles, 75 to 100,000 particles, 100 to 50,000 particles, 200 to 40,000 particles, 300 to 30,000 particles, 400 to 25,000 particles, or 500 to 20,000 particles. In other embodiments, the solid matrix material can have a plurality of particles ranging from 750 to 15,000 particles. In yet other embodiments, the solid matrix material can have a plurality of particles ranging from 25 to 500,000 particles, 50 to 500,000 particles, 75 to 500,000 particles, 100 to 500,000 particles, 200 to 500,000 particles, 300 to 500,000 particles, 400 to 500,000 particles, 500 to 500,000 particles, 1000 to 500,000 particles, 1500 to 500,000 particles, 2500 to 500,000 particles, or 5000 to 500,000 particles. In still other embodiments, the solid matrix material can have a plurality of particles ranging from 750 to 15,000 particles. In even other embodiments, the number of particles of the solid matrix material can be any sub-range or combination of these amounts.

The solid matrix material can have any number of spheres (or beads). In some embodiments, the solid matrix material comprises a plurality of spheres (or beads) ranging from 2 to 10 million spheres (or beads), 2 to 1 million spheres (or beads), 10 to 500,000 spheres (or beads), 25 to 250,000 spheres (or beads), 50 to 200,000 spheres (or beads), 75 to 100,000 spheres (or beads), 100 to 50,000 spheres (or beads), 200 to 40,000 spheres (or beads), 300 to 30,000 spheres (or beads), 400 to 25,000 spheres (or beads), or 500 to 20,000 spheres (or beads). In other embodiments, the solid matrix material can have a plurality of spheres (or beads) ranging from 750 to 15,000 spheres (or beads). In yet other embodiments, the solid matrix material can have a plurality of spheres (or beads) having from 25 to 500,000 spheres (or beads), 50 to 500,000 spheres (or beads), 75 to 500,000 spheres (or beads), 100 to 500,000 spheres (or beads), 200 to 500,000 spheres (or beads), 300 to 500,000 spheres (or beads), 400 to 500,000 spheres (or beads), 500 to 500,000 spheres (or beads), 1000 to 500,000 spheres (or beads), 1500 to 500,000 spheres (or beads), 2500 to 500,000 spheres (or beads), or 5000 to 500,000 spheres (or beads). In still other embodiments, the number of spheres of the solid matrix material can be any sub-range or combination of these amounts.

The solid matrix material can comprise spheres (or beads) with any diameter. In some embodiments, the spheres (or beads) can have a diameter ranging from about 0.01 mm to about 10 mm, about 0.02 mm to about 5 mm, about 0.03 mm to about 4 mm, about 0.04 mm to about 3 mm, about 0.05 mm to about 2 mm, about 0.05 mm to about 1 mm, about 0.05 mm to about 0.8 mm, about 0.05 mm to about 0.6 mm, or about 0.05 mm to about 0.4 mm, or about 0.05 mm to about 0.3 mm. In other embodiments, the spheres (or beads) can have a diameter ranging from 0.08 mm to about 0.2 mm. In yet other embodiments, the solid matrix material can comprise spheres (or beads) having a diameter of about 0.1 mm. In still other embodiments, the diameter of the spheres (or beads) of the solid matrix material can be any sub-range or combination of these amounts.

The solid matrix can be granular with any size. In some embodiments, the grains can have a US mesh size in the range of about 3.5 to about 632, about 10 to about 632, about 20 to about 500, about 30 to about 400, about 40 to about 325, or about 50 to about 200. In other embodiments, the solid matrix is granular and of a US mesh size of about 50, about 60, about 70, about 80, about 100, about 120, about 140, about 170, about 200, about 230, about 270, about 325, or about 400. In still other embodiments, the size of the grains of the solid matrix material can be any sub-range or combination of these amounts.

The solid matrix material can be made of any material that allows nematodes to move more efficiently in liquid media, thus providing a solid matrix within the liquid media that supports movement of the nematodes to different micro-environments e.g., that may be more conducive to their health. In some embodiments, the material can be selected from plastic, sand, diatomaceous earth, silicon, glass, inert carbon, zirconia, and combinations thereof. In other embodiments, the material comprises inert carbon. In yet other embodiments, the material comprises glass.

The solid matrix material can comprise a plurality of beads. In some embodiments, the beads are selected from Sepharose beads, Affigel beads, silica beads, zirconium beads, and combinations thereof. In some configurations, the beads are derivatized. Indeed, the beads can be derivatized with a polymer, a carbohydrate, a protein, an antibody, an antigen, an organic molecule, or combinations thereof. In some embodiments, the solid matrix material can comprises glass beads having a diameter in the range of about 0.08 mm to about 0.12 mm.

The solid matrix material can be obtained commercially or manufactured using process. Glass beads of various diameters for use as a solid matrix material as described herein are commercially available from e.g., Biospec Product Inc. Bartlesville, Okla. (e.g., catalog no. 11079101 for 0.1 mm diameter glass beads). Other types of beads include, but are not limited to, Sepharose (GE Healthcare), Affigel (Bio-Rad), and Agarose (many suppliers such as GoldBio, Agarose Beads Technologies). As well, various beads are commercially available (VWR; Bio-Rad, etc.) and may also be derivatized with chemicals, carbohydrates, polymers, proteins, antibodies and similar molecules.

The solid matrix material can be contained in a liquid medium to obtain the composite culture composition. The liquid medium can contain any components sufficient for growing or maintaining a plurality of nematodes. In some embodiments, the liquid media is selected from M9, S-basal, S-medium, S-complete, CeMM, and combinations thereof. In other embodiments, the liquid medium is water-based and contains any buffering agent, such as a phosphate buffer.

The liquid medium can contain trace metals. Any trace metal sufficient for growing, culturing or maintaining nematodes for chronic periods of time can be included in the liquid medium, including ferrous iron, manganese(II), zinc(II), cupric copper or combinations thereof. Other metal ions that can be used in the medium include, but are not limited to, calcium(II) and magnesium(II). In some embodiments, the liquid medium has nutrients sufficient for growing, culturing or maintaining nematodes for chronic periods of time. In some implementations, these chronic periods of time can be more than 20 hours and up to or longer than 10 days. Examples of these nutrients include cholesterol, or an inactivated or dead (or has both live and dead bacteria) bacterial preparation. The bacterial preparation can include inactivated or dead Bacillus subtilis, Bacillus mycoides or Bacillus soli. In some aspects, the bacterial preparation comprises inactivated or dead E. coli, including inactivated or dead E. coli OP50. Some methods of preparing nematode nutrients that can be used are described in Abada et al. Mol Cells. 2009 September; 28(3):209-13; Shtonda et al. J Exp Biol. 2006 January; 209(Pt 1):89-102). In other aspects, the inactivated bacteria can be inactivated by freezing, UV light, or heat.

The ratio of solid matrix material to the liquid medium can be adjusted for any desired property. In some embodiments, the amounts of these materials are such that when mixed together and settled in a receptacle, the liquid medium covers the solid matrix material and is no greater than about 2 times the height of the solid matrix material. In other embodiments, the liquid medium covers the solid matrix material and is no greater than about 1.8, about 1.6, about 1.4, about 1.3, about 1.2, or about 1.1 times the height of the solid matrix material. In yet other embodiments, the liquid medium covers the solid matrix material and is about 1 mm to about 5 mm above the solid matrix material. In still other embodiments, the liquid medium completely covers the solid matrix material and is about 1 mm above the solid matrix material. In one aspect, the liquid medium completely covers the solid matrix material such that about 10% or less of the liquid is above the solid matrix material. In the embodiments where the solid matrix material comprises glass beads with a diameter of about 0.08 mm to about 0.12 mm, the ratio of weight of the liquid medium to weight of glass beads per well can range from about 1:3 to about 2:1, from about 1:2 to about 1:1, or from about 3:5 to about 4:5 liquid medium to beads. In still other embodiments, the ratio can be any sub-range or combination of these amounts. In even other embodiments, the glass beads can be added to the liquid media such that about 10% or less of liquid remains above the beads.

In some configurations, the composite culture composition (or medium) further comprises a plurality of nematodes. The nematodes can be a C. elegans nematodes, genetically engineered or transgenic C. elegans nematodes, or genetically engineered or transgenic C. elegans having a transgene which is a promoter-reporter construct where the reporter encodes a fluorescent or luminescent protein and wherein the promoter is a promoter of a C. elegans gene induced in response to the organism's exposure to a toxin or stress. The nematodes can also be a mixture of combination of these.

In some embodiments, the composite culture composition (or medium) can be contained in any object or container having a plurality of receptacles. In these embodiments, one or more of the receptacles can contain any desired quantity of the solid matrix material and/or a quantity of the composite culture composition. In some embodiments, the container can have 2 to 10,000 receptacles, 2 to 9,600 receptacles, 2 to 5,000 receptacles, 2 to 3,456 receptacles, 2 to 1600 receptacles, 2 to 1,536 receptacles, 2 to 500 receptacles, 2 to 384 receptacles, 2 to 150 receptacles, 2 to 96 receptacles, 2 to 50 receptacles, 2 to 48 receptacles, or 2 to 24 receptacles or 2 to 12 receptacles. In other embodiments, the receptacles can be wells and so the container comprises a microtiter plate having 6 wells, 12 wells, 24 wells, 48 wells, 96 wells, 384 wells, or 1536 wells. In still other embodiments, the receptacle and/or wells can be any sub-range or combination of these amounts.

The container can contain either just the solid matrix material or the composite culture composition. In some configurations, the container can contain the solid matrix material in at least about 10%, about 20%, about 30%, about 40%, about 50%, about 60%, about 70%, about 80%, about 90%, about 95%, or in 100% of the receptacles (or wells). In other configurations, the container contains the composite culture composition in at least about 10%, about 20%, about 30%, about 40%, about 50%, about 60%, about 70%, about 80%, about 90%, about 95%, or in 100% of the receptacles (or wells). In still other embodiments, these percentages can be any sub-range or combination of these amounts.

The container can be selected from any number of containers or objects. In some embodiments, the container can be an Eppendorf tube, 0.6 ml microtube, 1.5 ml microtube, PCR tube, PCR tube strips, glass test tube, or plastic test tube, or a combination thereof. These containers can be obtained commercially from suppliers such as Millipore (Billerica, Mass.) or Pall Corporation (Ann Arbor, Mich.) or manufactured to specification.

Regardless of the container selected, one or more of the wells or receptacles of the container can contain a plurality of nematodes. In some configurations, a microtiter plate having 2 or more wells can be used wherein at least one well has a control population of a plurality of nematodes and at least one well has a test population of nematodes. The control population of nematodes can be treated with a vehicle while the test population can be treated with an agent. For example, the control population can be treated with the same vehicle that is used for treating the test population with the agent (agent plus vehicle). In some instances, 1, 2, 3, 4, 5, 6, 7, 8, 9, or 10 or more of the wells have a plurality of nematodes.

In some embodiments, one or more of the nematodes can be C. elegans. In these embodiments, 1, 2, 3, 4, 5, 6, 7, 8, 9, or 10 or more of the wells have a plurality of nematodes which comprise a plurality of C. elegans. For example, the plurality of nematodes can comprise a plurality of genetically engineered or transgenic C. elegans. In another example, the plurality of nematodes can comprise a plurality of genetically engineered or transgenic C. elegans having a transgene which is a promoter-reporter construct wherein the reporter encodes a fluorescent or luminescent protein and wherein the promoter is a promoter of a C. elegans gene induced in response exposure to a toxin or stress. The strains of C. elegans for use as nematodes can be any strain of C. elegans, including those described herein. Other strains include, but are not limited to, those that can be acquired from the Caenorhabditis Genetics Center at the University of Minnesota, St. Paul.

In one example of a container and its configuration, the composite culture composition is contained in a 96 well microtiter plate. Two (or 10 or 50) or more of the wells each contain first, about 50 to about 160 mg of glass beads with each bead having a diameter of ranging from about 0.08 mm to about 0.12 mm and second, about 60 to about 150 microliters of liquid media having inactivated bacteria, and optionally a test agent. The wells can further comprise from 50 to 600 nematodes. Alternatively, each well can contain about 130 mg of about 0.1 mm diameter glass beads, about 70 microliters of liquid media containing bacteria (which can comprise e.g., inactivated bacteria or live bacteria), optionally a test agent in 10 microliters of M9 media, and 80 to 500 nematodes.

The container containing the solid matrix material and optionally the liquid media can be used in methods to assess toxicology or the effects of an agent on transgenic nematodes as described in U.S. patent application Ser. No. 13/476,790 filed May 21, 2012, the entire disclosure of which is incorporated herein. In these methods, the conditions for maintaining the liquid media and the nematodes like C. elegans are known in the art. See e.g., www.wormbook.org section 5 or Szewczyk et al. BMC Biotechnology (2003) 3:19.

Any liquid media can be used in these methods to assess toxicology or the effects of an agent on transgenic nematodes. Examples of some liquid media that can be used include S-Basal, S-complete, S-Medium, M9, and combinations thereof. The S-Basal liquid media can prepared with 5.85 g NaCl, 1 g K₂HPO₄, 6 g KH₂PO₄, 1 ml cholesterol (5 mg/ml in ethanol), H₂O to 1 L and sterilized by autoclaving. 1 M Potassium citrate pH 6.0 is prepared with 20 g citric acid monohydrate, 293.5 g tri-potassium citrate monohydrate, H₂O to 1 L and sterilized by autoclaving. Trace metals solution is prepared with 1.86 g disodium EDTA, 0.69 g FeSO₄.7H₂O, 0.2 g MnCl₂.4H₂O, 0.29 g ZnSO₄.7H₂O, 0.025 g CuSO₄.5H₂O, H₂O to 1 L and sterilized by autoclaving. The trace metal solution is stored in the dark. 1 M CaCl₂ 55.5 g CaCl₂ in 1 L H₂O and is sterilized by autoclaving. The S Medium liquid media can be prepared with 1 L of S Basal, 10 ml 1 M potassium citrate pH 6, 10 ml trace metals solution, 3 ml 1 M CaCl₂, and 3 ml of 1 M MgSO₄. The components are added using sterile technique and this solution is not autoclaved. S-medium can be made more concentrated, these concentrations are the intended final concentrations of the liquid medium. M9 medium is prepared with 3 g KH₂PO₄, 6 g Na₂HPO₄, 5 g NaCl, 1 ml 1 M MgSO₄, H₂O to 1 L and sterilized by autoclaving. The M9 liquid media can be made more concentrated, these concentrations are the intended final concentrations of the liquid medium. In some embodiments, the container, vessel or receptacle containing the solid matrix material and the liquid medium can be used in methods for growing or maintaining a plurality of nematodes or improving their viability. These methods comprise the step of contacting a plurality of nematodes with liquid medium comprising a solid matrix material and one or more nutrients under conditions sufficient to culture (promote the growth of or maintain) a plurality of nematodes or improve their viability. In particular, these conditions include maintaining the nematodes at any temperature that promotes the growth of or maintains the nematodes. Furthermore, the amount of time that the nematodes are subjected to these conditions can be any amount of time as long as it promotes the growth or maintains the nematodes. The times and temperatures are chosen to suit the purpose of maintaining or promoting the growth of the nematodes. For example, a chronic assay, in some instances may run from 1 to 10 days and the nematode or nematodes, like C. elegans, in some instances are maintained at a temperature in the range of 15° C. to about 25° C.

In some instances, the conditions sufficient to culture (including promote growth of or maintain) a plurality of nematodes or improve their viability include maintaining the nematodes at a temperature in the range of about 10° C. to about 29° C. In other instances, the culture (e.g., solid matrix material immersed in liquid media) containing a nematode or plurality of nematodes can be maintained at a temperature in the range of about 15° C. to about 25° C. As well, the culture (e.g., solid matrix material immersed in liquid media) containing a nematode or plurality of nematodes, in some instances, can be maintained for about 10 minutes to about 10 days or more, from about 1 day to about 10 days, from about 1 day to 9 days, from about 1 day to about 8 days, or from about 1 day to about 7 days.

The culture (e.g., solid matrix material immersed in liquid media) containing a nematode or plurality of nematodes like C. elegans can also be maintained at about 10° C. to about 15° C. or from about 25° C. to about 29° C. for about 1 hour to about 24 hours. Brief exposure of the culture (e.g., solid matrix material immersed in liquid media) containing a nematode or plurality of nematodes such as C. elegans for less than about 1 hour can occur at about 4° C. to about 10° C. or about 30° C. to about 37° C. Exposure at times less than about 1 hour is possible for temperatures of about 4° C. to about 37° C. These incubations allow maintenance to occur with the C. elegans nematode life cycle which can be about 5 days at about 15° C., about 3 days at about 20° C., and about 2 days at about 25° C. in some embodiments. As the skilled artisan is aware, different nematodes (e.g., other than C. elegans) may have different optimal or acceptable temperature ranges for growth, maintenance, culturing and/or survival, and the above ranges can be modified to suit the particular species of nematode. In some implementations the nematodes are maintained a particular temperature within the ranges disclosed here which is a substantially constant temperature. The constant temperature can be e.g., a specific temperature ±about 3° C., ±about 2° C., ±about 1° C., ±about 0.5° C., or ±about 0.2° C. In still other embodiments, these times and temperatures can be any sub-range or combination of these amounts.

In some instances of the embodiments described herein, the culture or compositions comprise one or more nutrients. Accordingly, the one or more nutrients can comprise a nutrient which is food or nematode food which is provided in any amount that is sufficient for the nematode or plurality of nematodes. The nematode food is generally provided in the range of about 0.05% to about 5.0%. Typically, the food is inactivated bacteria. Inactivation refers to treatment of the bacteria so that it no longer grows or is killed. For example, bacteria, such as E. coli, can be killed by freezing the pellet obtained from a spinning down a liquid culture of the bacteria. The skilled artisan is familiar with techniques for growing bacteria and preparing them as a food source for nematodes. The amount of food can be any amount or is from about 0.05% to about 5%, about 0.1% to about 3%, or about 0.1% to about 2.5% or about 0.3% where these amounts are calculated as volume of pellet to volume of pellet and added solution. For instance, a 10 ml pellet with 90 ml buffer added gives a 10% suspension. Depending on the nature of the experiment or the purpose for culturing the nematodes different amounts of food can be used and different sources of food can be used including different strains of E. coli (e.g., HB101 or OP50) or different bacteria such as various species of Bacillus. Other sources of food can be used for species of nematodes that do not eat or grow on bacteria as known by the skilled artisan.

In these methods, the plurality of nematodes can range from 2 to 100,000 nematodes, 5 to 50,000 nematodes, 10 to 40,000 nematodes, 20 to 30,000 nematodes, 30 to 20,000 nematodes, 40 to 15,000 nematodes, 50 to 10,000 nematodes, 100 to 10,000 nematodes, 200 to 10,000 nematodes, 300 to 10,000 nematodes, 400 to 10,000 nematodes, or 500 to 10,000 nematodes. In some embodiments, the plurality of nematodes can range from 25 to 2000, 25 to 1500, 25 to 100 or 25 to 750 nematodes. In still other embodiments, the plurality of nematodes can be any sub-range or combination of these amounts.

The age of the nematodes can be synchronized or unsynchronized. In some embodiments, the numbers of nematodes for synchronized cultures can range from 1 to 10000, to 5000, 50 to 2500, or 100 to 1000 nematodes. In other embodiments, the number of nematodes for asynchronous cultures can range from 10 to 100,000, 100 to 75,000, 200 to 50,000, 300 to 40,000, 400 to 25,000, or 500 to 10,000. In still other embodiments, the number of nematodes can be any sub-range or combination of these amounts.

In these methods, the plurality of nematodes can comprise a plurality of C. elegans. In some embodiments, the plurality of nematodes can comprise a plurality of genetically engineered or transgenic C. elegans. In other embodiments, the plurality of nematodes can comprise a plurality of genetically engineered or transgenic C. elegans having a transgene which is promoter reporter construct wherein the reporter encodes a fluorescent or luminescent protein and wherein the promoter is a promoter of a C. elegans gene induced in response exposure to a toxin or stress. In yet other embodiments, the plurality of nematodes can be one or more, two or more, 3 or more, 4 or more, or 5 or more populations of representative transgenic nematodes (e.g., C. elegans), as described in U.S. patent application Ser. No. 13/476,790 filed May 21, 2012, the entire disclosure of which is incorporated herein by reference.

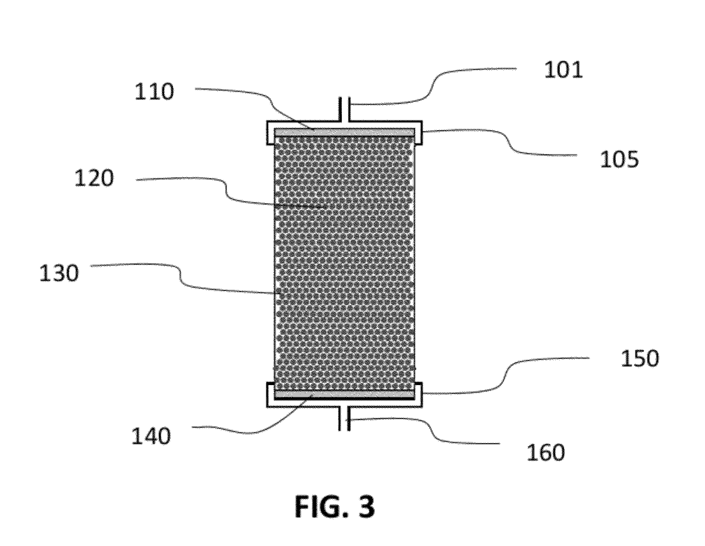

In some embodiments, a bioreactor can be used for nematode growth. In these embodiments, the bioreactor contains at least solid matrix material immersed in a liquid medium. An exemplary bioreactor is shown in FIG. 3 where bioreactor has an inlet port, 101, that allows delivery of liquid medium or buffer (e.g., M9, S-Basal, S-complete etc.) to the bioreactor vessel 130. The inlet port, 101, may have a fitting or coupling that can attach to an inlet line that allows for or provides for delivery of liquid media or buffer. The liquid medium or buffer can be delivered by any mechanism for fluid delivery, including via a pump (e.g., pump or peristaltic pump). The bioreactor contains a lid or cap, 105, that adjoins or is part of the bioreactor vessel 130. The lid or cap, 105, may be removable so as to allow access to the chamber of the bioreactor vessel for e.g., removing or adding contents to the chamber of the bioreactor vessel.

As shown in FIG. 3, the bioreactor can have a filter, membrane or mesh, 110, that prevents escape of the nematodes or solid matrix material from the bioreactor but allows for the delivery of liquid medium or buffer. The bioreactor is designed to contain a solid matrix material, 120, that comprises a plurality of particles (e.g., particles like glass beads). Another filter, membrane or mesh, 140, may be present that prevents escape of nematodes and solid matrix material from the bioreactor vessel but allows for the exit or outlet of liquid medium or buffer. Another cap or lid, 150, is adjoined to or an integral part of the bioreactor vessel which may be removable or may be an integral part of the bioreactor vessel. An outlet port, 160, can also be a part of the bioreactor that allow outlet or exit of the liquid media or buffer after it has passed from the bioreactor. The outlet port, 160, can drain into a waste container or drain or be collected to analyze or utilize its contents.

The bioreactor illustrated in FIG. 3 contains at least a solid matrix material immersed, interspersed, or bathed in a liquid medium. The liquid medium can contain at least a buffering agent, such as a phosphate buffer. The bioreactor can contain a plurality of nematodes. The bioreactor can also contain a temperature regulation device to maintain and control the temperature of the bioreactor. The bioreactor has a flow regulator that allows for and controls the passage of a liquid (e.g., aqueous) through the bioreactor. Thus, the bioreactor can be configured to collect material produced (e.g., excreted or secreted) by the plurality of nematodes (e.g., protein, antibody, or other product) or the bioreactor can be configured to collect a plurality of nematodes. In some configurations, the bioreactor has the capacity to contain a volume of about 10 milliliters or more of composite culture composition. In other configurations, the bioreactor has the capacity to contain a volume of about 1 L or more, about 5 L or more, or about 10 L or more of composite culture composition or contains about 1 L, about 5 L or more, or about 10 L or more of the composite culture composition.

The container containing the solid matrix material and optionally the liquid media can be used in methods for improving nematode viability in liquid culture. These methods comprise the step of contacting a nematode or population of nematodes in need of improved viability with a liquid culture medium having a solid matrix (e.g., composite culture composition). The nematode can be a genetically engineered nematode or genetically engineered population of nematodes. In some aspects, the nematode or population of nematodes has reduced viability wherein the reduced viability is induced by a mutation or an external agent or both. In other aspects, the external agent comprises a toxin, a drug or pharmaceutical preparation, an environmental poison, or combinations thereof. These methods include using a plurality of nematodes as described herein.

The solid matrix material described herein can be used in assay methods. These methods comprise the step of providing a solid matrix material immersed, interspersed, or bathed in a liquid medium and either (1) treating a nematode or plurality of nematodes with an agent and introducing a nematode or plurality of nematodes treated with an agent to the solid matrix material immersed, interspersed or bathed in liquid medium or (2) introducing a nematode or plurality of nematodes to the solid matrix material immersed in liquid medium to give a nematode culture and treating the nematode culture with an agent. These methods can also comprise maintaining or growing the nematode culture for a time sufficient to allow for the agent to affect the nematode culture. In some aspects, the time sufficient can be at least about 1 hour, at least about 2 hours, at least about 3 hours, at least about 4 hours, at least about 5 hours, at least about 6 hours, at least about 8 hours, at least about 10 hours, at least about 12 hours, at least about 16 hours, at least about 20 hours, or at least about 24 hours. In other aspects, the time sufficient is at least about 1 day, at least about 2 days, at least about 3 days, at least about 4 days, at least about 5 days, at least about 6 days, at least about 7 days, at least about 8 days, at least about 9 days, or at least about 10 days. In yet other aspects, this time can be any combination or sub-range of these amounts.

In some embodiments, these methods also comprise determining, monitoring, or measuring the effect of the agent on the nematode culture after it has been maintained for a time sufficient to allow for the agent to affect the nematode culture. In some aspects, determining, monitoring or measuring the effect of the agent on the nematode culture comprises determining the number of live nematodes, determining the number of dead nematodes, determining an increase or decrease in the biomass in the nematodes, and determining the level of one or more reporter genes in the nematodes. These methods can also involve a fluorescence reporter test. The agent can be an RNAi interference compound. The method can be configured for high-throughput drug screening, for high-throughput toxicity screening, for biochemical activity test, for mass spectrometry tests, for flow-cytometry tests, and/or for movement activity tests. In one aspect, the plurality of nematodes is a plurality of C. elegans. These methods can use the plurality of nematodes as described herein.

Some methods use a 96 well microtiter plate that has per well about 270 mg glass beads (0.1 mm diameter); 30 μl of 3× liquid medium (food at 3%); 30 μl of 3× final nematode concentration; and 30 μl of solubilized test compound at 3× final concentration. Not all wells of the 96 well microtiter plate have to be used. For example, 5, 10, 15, 20, 30, 40, 50, or 60 or more wells are used for test compound whereas 1, 2, 3, 4, or 5 or more wells of the same plate are used for controls. Each well can have 25 to 750 nematodes, 25 to 650 nematodes, or 25 to 550 nematodes. In some embodiments each well can have 1 to 25 nematodes.

In some embodiments, a kit can be used for nematode growth, maintenance or culturing. In these embodiments, the kit comprises an object or container having a plurality of receptacles. The kit can have any quantity of solid matrix material. The kit can also have a liquid media comprising at least one buffering agent, such as a phosphate based buffer. The liquid media can comprise trace minerals or metals sufficient for maintaining the nematode over specific period of time, for example, about 1 day, 5 days, or even 10 days or more. The liquid media can be sterile or unsterile. The kit can comprise one or more nutrients. The nutrient or liquid medium can be reconstituted by the addition of water or sterile water. Thus, the kit can contain water or sterile water. In some configurations, the kit contains a device for dispensing or measuring a unit volume of solid matrix material, nutrient, liquid media or water wherein the unit volume is the amount sufficient for each receptacle of the object or container in the kit. The kit can also contain one or more reagents for nematode identification. Examples of reagents for nematode identification include primers, probes, and/or reagents for PCR-based identification of nematodes.

Alternatively, the kits can contain a gas-permeable floor culture system. In these embodiments, the kit comprises an object or container having a plurality of receptacles wherein one or more receptacles have a gas-permeable floor. The gas permeable floor can comprise a mesh floor, such as a 40 micron mesh floor. The kit can contain a liquid media comprising at least one buffering agent, such as a phosphate buffer. In these embodiments, the liquid media can comprise trace minerals or metals sufficient for maintaining the nematode over specific period of time, for example, about 1 day, 5 days, or even 10 days or more. The liquid media can be sterile or unsterile. The kit can comprise one or more nutrients. The nutrient or liquid medium can be reconstituted by the addition of water or sterile water. Thus, the kit can contain water or sterile water.

The viability of nematodes in liquid when the nematodes are compromised for mobility can be also improved or maintained. These methods comprise the step of contacting a locomotion-compromised nematode or population of nematodes in need of maintaining or improved viability with a liquid culture medium in a vessel with a gas-permeable floor. The nematodes can be wild-type with depressed mobility, slow moving nematodes, or sedentary nematodes. In some aspects, the nematodes are wild-type but unable to move normally due to exposure to an agent, temperature, gas or other condition that depresses mobility. The nematodes can carry a genetic mutation or mutations that interfere with mobility. The liquid medium can have a sufficient viscosity to depress mobility.

In these embodiments, the gas-permeable floor comprises a mesh that allows gas but not liquid to cross the floor substantially spontaneously. The gas-permeable floor can comprise a mesh floor having a mesh in the range of about 10 to about 200, about 10 to about 150, or about 20 to about 100 micron. In some configurations, the gas-permeable floor comprises a 20 micron mesh, a 40 micron mesh, a 60 micron mesh, or a 100 micron mesh. In other configurations, the mesh can comprise any combination or sub-range of these amounts.

In yet other configurations, the gas-permeable floor comprises a mesh that can be crossed by nematodes at the L1, L2, or L3 stage of development, but not at the L4 or adult stage of development. The vessel's gas-permeable floor can comprise a mesh through which nematodes at a particular stage of development can pass when the vessel is centrifuged. The gas-permeable floor can also comprise a membrane that allows gas but not liquid to flow spontaneously. The gas-permeable floor can also comprise a membrane that allows passage of certain gases but not others, such as a membrane that allows passage of carbon dioxide and oxygen but no other gases. In some configurations, the vessel contains a 6-well, 24-well, 96-well, or 384-well microtiter plate, wherein the floors of the wells are gas-permeable. The vessel can also contain a 96-well microtiter plate that has a floor comprising a 40 micron nylon mesh. The vessel can contain the plurality of nematodes as described herein.

These methods can enable motile nematodes to thrive in liquid media. But worms that are completely unable to move or that are severely compromised for locomotion may not thrive under the conditions of a culture containing a solid matrix immersed in a liquid medium. In these cases, worms are unable to move to local environments conducive to their health, despite the presence of a matrix that enables motile nematodes to prosper in liquid media. As a result, paralyzed worms exhibit slow growth, delayed development, and poor viability.

Thus, methods and compositions that enable growth of locomotion-compromised nematodes (e.g., paralyzed nematodes or nematodes that move slowly or are sedentary) in liquid media can be used. The nematodes can be any of the nematodes described herein. In these embodiments, nematodes (e.g., a population or plurality) can be grown in liquid media in wells of microtiter plates where the bottoms or floors of the microtiter wells comprise a mesh or a mesh material (see e.g., FIG. 4 or Example 3). One example comprises a microtiter plate (e.g., commercially available) fitted atop a receiver tray such that the mesh bottom of the microtiter wells are suspended above the floor of the receiver tray. The receiver tray can have an amount of water or another liquid in it to provide or maintain humidity, although the level of water or liquid is not in contact of with the gas permeable floor; that is, the level of water or another liquid is, e.g., sufficiently below the level of the gas-permeable floor to allow a layer of gas or air to be present in the receiver tray below the gas-permeable floor, therefore providing gas to transit the gas-permeable floor into the liquid medium. The gauge of the mesh can be sufficiently fine to prevent (or substantially limit) gravity-driven passage of aqueous media from the microtiter well into the receiver tray.

When paralyzed worms (e.g., nematodes including C. elegans) sediment to the bottom of the well, they settle against the mesh into a local environment where partial pressures of oxygen and carbon dioxide are virtually identical to the gaseous environment of the receiver tray. Since this gaseous environment is typically room air, the conditions simulate those found in conventional solid-media (e.g., agarose) configurations. Moreover, the position of the nematodes within the microtiter plate minimizes the possibility of nutritional limitation. Over time, bacteria in the medium sediment into the same local environment into which the worms have settled and thereby replenish bacteria consumed by the motionless worms. To demonstrate these embodiments, a Millipore Multiscreen-MESH 96-well microtiter plate was used with 40 μm nylon mesh. It was observed that severely paralyzed worms in liquid media exhibit growth rates and development times that were unexpectedly indistinguishable from those observed on conventional agarose media. In other embodiments, an oxygen- and carbon dioxide-permeable membrane can be used instead of a mesh form as the vessel floor. These embodiments share the ability of using vessels with gas-permeable floors to eliminate the deleterious effects on nematodes of immersion in the liquid medium.

The movement-impaired or movement-depressed nematodes can be normal nematodes e.g., wild-type or an otherwise normal-moving nematode strains that have been treated with an agent in an amount sufficient to impair movement or depress movement. Agents that impair or depress movement in nematodes include, but are not limited to, levamisol, aldicarb, ivermectin, sodium azide, phenoxypropanol, volatile anesthetics (halothane, enflurane, isoflurane, etc), PEG 20000 and glycerol in 1×PBS, nitrous oxide, ethanol, and combinations thereof. Movement impaired or depressed nematodes can also include those that have mutations that impair or depress movement including, but not limited to, unc-1 to unc-132 (including unc-2, unc-3, unc-4, unc-5, etc. to unc-132), ric-1 to ric-19 (including ric-2, ric-3, ric-4, ric-5, etc. to ric-19). Other locomotion-impaired or depressed nematodes are known in the art and/or can be prepared using known methods. See e.g., Sieburth et al. Nature. 2005 Jul. 28; 436(7050):510-17; Brenner S., Genetics 1974 May; 77(1):71-94; Maduro et al. Genetics 1995 November; 141(3):977-88.

In these embodiments, the culture composition comprises a solid matrix material with a plurality of particles which sits upon the gas-permeable floor. The solid matrix material can be optionally interspersed, immersed, or bathed in the liquid medium. In some configurations, the solid matrix material can be interspersed in the liquid media to give composite growth media wherein a population of nematodes can position themselves for optimal growth in response to one or more nutrient gradients. The liquid media can be in the buffers of M9, S-basal, S-complete, or CeMM. The solid matrix material can be comprised of glass beads of a size and weight sufficient to promote or enhance nematode growth when interspersed in a liquid medium. In some configurations, the glass beads can have a diameter of about 0.05 mm to about 2 mm. In other configurations, the glass beads can have a diameter of about 0.1 mm.

The methods and compositions described herein can be utilized for a variety of purposes including nematode identification (e.g., identification of nematodes from soil samples, which may be useful in agricultural applications), drug screening for human diseases using nematode models, screening for nematicides, screening for antihelminthics, toxicology, research and development, veterinary parasitology, etc. An exemplary nematode that can be used comprises C. elegans. Free living soil-inhabiting nematodes include herbivores, bacterivores, fungivores, omnivores, predators and those of unknown trophic habits. Parasitic nematodes can be used and include, but are not limited, to agriculturally important nematodes like root-knot nematodes and reniform nematodes and nematodes important in human health including hookworm, lungworm, pinworm, threadworm, whipworm, and eelworm. Examples of parasitic nematodes also include e.g., Trichinella spiralis, ascarids, Baylisascaris, Dirofilaria immitis, and Haemonchus contortus.

### EXAMPLES

### Example 1

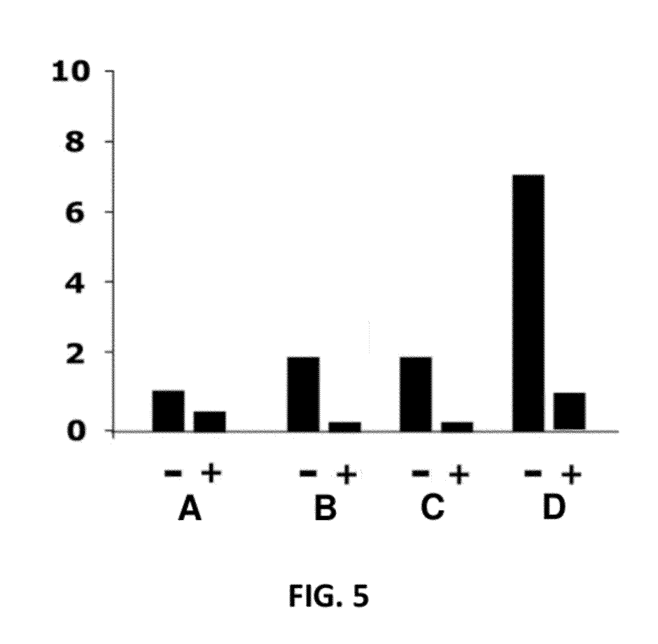

Effect of glass beads and E. coli on worm stress in liquid media (M9-based). The stress response of nematodes is measured by the hsp-16.41::GFP reporter transgene introduced into C. elegans using single site transgenesis technology. In samples without beads, induction of stress reporter is observed. In samples with beads stress response is suppressed. See FIG. 5. Assay conditions are 130 mg of beads (e.g., 0.1 mm diameter glass beads) in 90 μl of liquid medium (M9) that was used with and without 0.3% E. coli food. An asynchronous population of ˜100 nematodes was exposed to liquid culture in M9 buffer for 24 hrs, then allowed to recover for 4 hrs on HB101-seeded plates. Nematodes were scored (blinded study) for apparent intensity of fluorescence relative to heat shock (30° C., 1 hr). FIG. 5 illustrates the results of these experiments that show the effect of stress induced by E. coli preparation on C. elegans in liquid medium with (+) or without beads (−). (A) is the experiment run in 90 μL M9 media without food; (B) is the experiment run in 160 μL, M9 media without food; (C) is the experiment run in 90 μL M9 media with food (0.3% E. coli); and (D) is the experiment run in 160 μL M9 media with food (0.3% E. coli). The height of the bars represent stress as measured by induction of a green fluorescent protein GFP reporter gene driven by the C. elegans hsp-16.41 promoter relative to heat shock (which was set to 10) as measured by fluorescence. This result illustrates that excessive liquid medium in the well increases stress and that the presence of beads (e.g., 0.1 mm glass beads) reduces this stress as measured by the expression of a gene encoding a fluorescent reporter protein that is linked to a stress activated promoter.

### Example 2

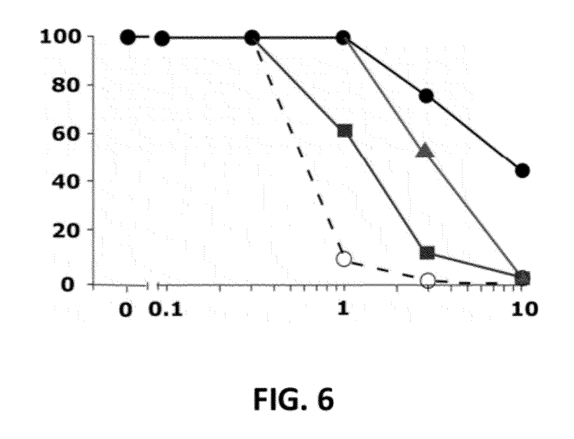

Effect of glass beads on E. coli tolerance in liquid growth conditions. Assay conditions are 0, 60, 130 and 200 mg of beads (e.g., 0.1 mm in diameter glass beads) in liquid at various concentrations of E. coli food (0, 0.1, 0.5, 1, 2 and 10%). Asynchronous nematode populations of ˜200 animals in a final volume of 150 μL M9 buffer with various E. coli concentrations were exposed to liquid culture and incubated for 24 hr at 22° C., after which 10% of the mixture was removed and assayed for worm survival. Nematodes were scored for percent survival by removing 10% of mixture and assaying numbers of worms that were alive and dead. FIG. 6 shows the effect of glass beads (e.g., 0.1 mm diameter) on E. coli tolerance in liquid growth conditions. The y-axis is percent nematode survival and the x-axis is percent bacterial content of media (E. coli strain HB101). FIG. 6 shows that increasing the amount of beads in the composite culture composition of the application increases nematode (e.g., C. elegans) viability (e.g., survival) where (∘) represents 0 mg beads, (▴) 60 mg beads, (▪) 130 mg beads, and () 200 mg beads.

### Example 3

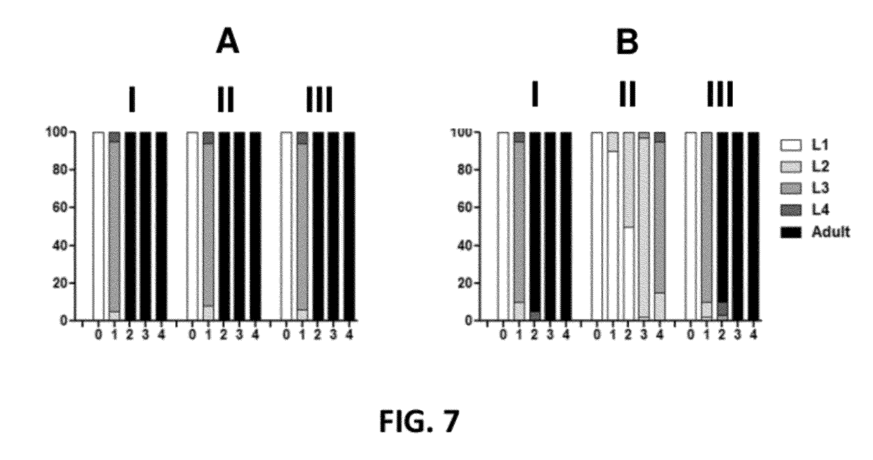

Growth comparisons of paralyzed nematodes in liquid and on solid media. Wild-type C. elegans or MT6039, a locomotion-deficient strain, were seeded at the L1 stage of development in 3 different formats: on conventional solid agarose media or into liquid media in microtiter wells distinguished by the presence of either glass beads or 40 micron nylon mesh floors (Millipore Cat #MANMN4010). In the case of the microtiter plate with mesh floors, the plate was placed into a receiver tray containing water to humidify the ambient air surrounding each well. The progress of larval development was monitored for each condition. Notably, development of wild-type worms is equivalent among the three formats, indicating the effectiveness of either liquid format to support growth of wild-type worms in liquid. In the case of the MT6039 strain, on the other hand, larval development is drastically delayed in the liquid format using glass beads. In the mesh floor format, however, these mutant worms develop virtually identically to those on solid media. Thus the gas-permeable membrane promotes the growth, maintenance, culturing and/or viability of locomotion depressed nematodes. See FIG. 7. FIG. 7A shows results according to developmental stage with wild-type C. elegans and FIG. 7B shows results according to developmental stage with locomotion impaired C. elegans strain MT6039. (I) is the respective nematodes grown on solid media (agarose), (II) is the respective nematodes grown in liquid media with beads, and (III) is the respective nematodes grown in liquid media on gas-permeable floors (as shown in FIG. 4). The y-axis in both FIG. 7A and 7B represents the percent distribution across developmental stage and the x-axis is the number of days (0, 1, 2, 3, and 4) from the start of the experiment with arrested L1 larvae. The developmental stage of the worms is reflected by the shading of the bars: L1 (no shading), L2 (light shading), L3 (intermediate shading), L4 (darker shading) and adult (darkest shading or black) The use of vessels with mesh or membranous floors allows normal development and viability of locomotion-deficient nematodes in liquid cultures. The use of gas-permeable vessel floors thus allows the use of existing automated and robotic liquid-handling technologies for work involving nematodes with reduced mobility. These liquid-handling technologies, in turn, enable a scale of experimentation with handicapped nematodes that would not be feasible otherwise. The value of such high-throughput experimentation is illustrated in the following example.

### Example 4

### High-Throughput Drug Screen

Example 4 describes a high-throughput screening application using unc-116, which is a C. elegans mutant that is deficient in the motor protein, kinesin-1. As with all C. elegans unc mutants, unc-116 worms are deficient in locomotion. An ortholog of unc-116 is kinesin-1 in humans which when mutated leads to a form of a hereditary spastic paraplegia (HSP; Am J Hum Genet 71:1189, 2002; Neurology 63:1108, 2004). Using 96-well microtiter plates with 40 micron mesh bottoms and appropriate liquid-handling equipment, a high-throughput screen of large compound libraries is conducted to identify rare chemicals that improve the mobility of unc-116 mutants. Libraries of hundreds of thousands of unique chemicals are tested using the mutant nematodes in e.g., 96-well plates using the gas-permeable floor as described herein. Chemicals that enhance the mobility of unc-116 mutants, detected by automated imaging of wells, are candidates for further testing as possible therapeutics for HSP. Notably, the robust viability of unc-116 worms in wells with gas-permeable floors enables those with pharmacologically rescued kinesin-1 function to display enhanced mobility.

Similar considerations apply to other unc or movement-compromised mutants. Indeed, using gas-permeable microtiter plates and available liquid-handling equipment, chemical libraries can be screened to identify agents that suppress any mutation that reduces the mobility of nematodes, thereby providing drug screening systems for important human diseases using a high-throughput whole organism approach.

In addition to any previously indicated modification, numerous other variations and alternative arrangements may be devised by those skilled in the art without departing from the spirit and scope of this description, and appended claims are intended to cover such modifications and arrangements. Thus, while the information has been described above with particularity and detail in connection with what is presently deemed to be the most practical and preferred aspects, it will be apparent to those of ordinary skill in the art that numerous modifications, including, but not limited to, form, function, manner of operation and use may be made without departing from the principles and concepts set forth herein. Also, as used herein, examples are meant to be illustrative only and should not be construed to be limiting in any manner.

What is claimed is:

1. A composite culture composition, comprising:
a solid matrix material comprising a plurality of particles; and
a liquid medium for nematode growth or maintenance.

2. The composition of claim 1 wherein the solid matrix material is selected from plastic, sand, diatomaceous earth, silicon, glass, inert carbon, silica, zirconia, and combinations thereof.

3. The composition of claim 1, wherein the plurality of particles have a shape selected from granular, spherical, cylindrical, polyhedron, and combinations thereof.

4. The composition of claim 1, wherein the plurality of particles comprise glass beads having a diameter between about 0.05 mm and about 0.5 mm.

5. The composition of claim 1, wherein the liquid medium comprises a phosphate buffer.

6. The composition of claim 1, wherein the liquid medium comprises a nutrient.

7. The composition of claim 1, further comprising a plurality of free-living or parasitic nematodes.

8. The composition of claim 1, further comprising a plurality of transgenic C. elegans having a transgene having a C. elegans promoter operably linked to a gene encoding a fluorescent or luminescent protein.

9. A method of growing, culturing, or maintaining a plurality of nematodes, comprising:
providing a composite culture composition having a solid matrix material combined, immersed or interspersed in a liquid medium containing a buffering agent; and
contacting a plurality of nematodes with the composite culture composition.

10. The method of claim 9, wherein the liquid medium comprises a nutrient.

11. The method of claim 9, wherein the liquid medium comprises a nutrient which is inactivated or live bacteria.

12. The method of claim 9, wherein the solid matrix material comprises a plurality of spheres.

13. The method of claim 12, wherein the plurality of spheres comprise glass beads having a diameter ranging from about 0.05 mm to about 0.5 mm.

14. The method of claim 9, wherein the providing and contacting are carried out in a microtiter plate.

15. The method of claim 9, wherein the plurality of nematodes comprises a plurality of free-living or parasitic nematodes.

16. The method of claim 9, wherein the plurality of nematodes comprises a plurality of transgenic C. elegans.

17. An apparatus having a gas permeable floor, the apparatus containing a liquid medium having components sufficient for culturing a plurality of nematodes.

18. The apparatus of claim 17, further comprising a plurality of locomotion-depressed nematodes.

19. The apparatus of claim 17, wherein said container or vessel is a microtiter plate.

20. The apparatus of claim 17, wherein the gas permeable floor comprises a 20, 40, or 60 micron mesh.

21. A method of culturing a plurality of nematodes wherein the nematodes are locomotion-depressed, said method comprising contacting or combining a plurality of locomotion-depressed nematodes with a liquid medium wherein the liquid medium sits or rests in a vessel or container having a gas-permeable floor under conditions sufficient for culturing said plurality of locomotion-depressed nematodes.

22. The method of claim 21, wherein said plurality of locomotion-depressed nematodes are a plurality of C. elegans treated with an agent that depresses locomotion or having a mutation that depresses locomotion compared to wild-type.

23. The method of claim 21, wherein said vessel or container is a microtiter plate.

24. The method of claim 21, wherein the method grows, obtains, maintains, promotes the growth of, or promotes survival of the plurality of nematodes.
